(19) United States
(12) Patent Application Publication
(10) Pub. No.: US 2008/0228046 A1
(43) Pub. Date: Sep. 18, 2008

(54) HEALTH CARE SUPPORT SYSTEM

(75) Inventors: Kouki FUTATSUYAMA, Anjo-shi (JP); Shinji NANBA, Kariya-shi (JP); Kenichi YANAI, Nisshin-shi (JP); Taiji KAWACHI, Kariya-shi (JP)

Correspondence Address: POSZ LAW GROUP, PLC, 12040 SOUTH LAKES DRIVE, SUITE 101, RESTON, VA, 20191, US

(73) Assignee: DENSO CORPORATION, Kariya-city (JP)

(21) Appl. No.: 12/048,369

(22) Filed: Mar. 14, 2008

(30) Foreign Application Priority Data
Mar. 16, 2007 (JP) 2007-068950

(51) Int. Cl. A61B 5/00

(52) U.S. Cl. 600/301

(57) ABSTRACT

The present invention provides a health care support system that can give appropriate advice depending on a situation.

At first step, whether a driver is seated is checked using a signal from a pressure sensor. At second step, whether the driver is in a resting state is checked using heart rate and the like. At third step, physical information is acquired. At forth step, a health status is judged based on the measured pieces of physical information and the like. At fifth step, activity status information is acquired. At sixth step, an activity status is judged based on the activity status information. At seventh step, an appropriate piece of advice for the driver is selected based on a judgment result of the health status and a judgment result of the activity status. At eighth step, the selected piece of advice is displayed on a displaying section and audibly outputted.

### CROSS-REFERENCE TO RELATED APPLICATION

This application is based on and claims the benefit of priority from earlier Japanese Patent Application No. 2007-68950 filed Mar. 16, 2007, the description of which is incorporated herein by reference.

### BACKGROUND OF THE INVENTION

1. Technical Field of the Invention

The present invention relates to a health care support system that is mounted on an automobile, determines a health status of an occupant, and gives advice and the like related to lifestyle-related diseases based on the determined health status.

2. Description of the Related Art

In recent years, lifestyle-related diseases, such as obesity, high blood pressure, and diabetes, have become widespread. There is an increasing need for the prevention of these lifestyle-related diseases. A first step in preventing the lifestyle-related diseases is to measure body weight, body fat percentage, blood pressure, and the like frequently that are closely related with the lifestyle-related diseases and re-examine lifestyle based on the measured results.

To deal with the above-described lifestyle-related diseases, body weight, body fat percentage, blood pressure, and the like closely related with the lifestyle-related diseases are preferably frequently measured. However, these measurements not only require time, but are also required to be performed frequently over a long period of time. As a result, such a continual measurement is complicated and difficult for a person trying to determine his health status.

Therefore, a device has been proposed that can give advice related to the lifestyle-related diseases and the prevention of these diseases without requiring complicated continual measurements as disclosed in Japanese Patent Application Laid-Open Publication No, 2006-247175, for example.

However, a health status with regard to the lifestyle-related diseases changes over a long period of time, and changes do not appear over a few days, even when improvement measures are taken. Therefore, the person may become tired of receiving the same piece of advice and his/her motivation may decrease. Timing of the advice is also a problem. For example, if the person receives advice regarding a lifestyle-related disease before driving, the person cannot immediately follow the advice.

### SUMMARY OF THE INVENTION

The present invention has been achieved in light of the above described issued. An object of the present invention is to provide a health care support system that can give suitable advice for a specific situation.

An invention according to claim 1 is a health care support system that is mounted on an automobile and supports the health status of an occupant. The health care support system includes a physical information detecting means, a health status judging means, an activity status detecting means, an activity status judging means, an advice selecting means, and a notification controlling means.

The physical information detecting means detects a physical status of the occupant as physical information. The health status judging means judges the health status of the occupant from the physical information. The activity status detecting means detects activity information indicating an activity status of the occupant.

The activity status judging means judges the activity status of the occupant from the activity information. The advice selecting means selects a piece of advice to be given to the occupant based on a judgment result acquired by the health status judging means and a judgment result acquired by the activity status judging means. The notification controlling means drives a notifying means (such as a monitor or a speaker) and gives notification of the piece of advice.

In the present invention, various pieces of advice are given to the occupant based on the analysis of the health status and the judgment result of the activity status of the occupant. Therefore, the occupant can respond appropriately based on the advice, thereby having a very positive effect on health management.

In particular, in the present invention, a piece of advice can be given depending on an activity status at, for example, a location of the occupant. Therefore, a remarkable effect of allowing an appropriate piece of advice to be given at an appropriate timing can be achieved.

Here, the physical information detecting means and the activity status detecting means can be actualized by, for example, sensors, as described hereafter. The health status judging means, the activity status judging means, an electronic controlling device, such as a computer, can actualize the advice selecting means, and the notification controlling means.

In an invention according to claim 2, the physical information is at least one kind among body temperature, blood pressure, heart rate, pulse wave information, body weight, blood sugar level, body fat, and height. The physical information includes at least one of either a medical history of the occupant or a medical history of a family member. The physical information further includes a medical examination result of the occupant.

In an invention according to claim 3, at least one kind among an electrode attached to a steering wheel, a pulse wave sensor, an electrode attached to a seat, a pressure sensor attached to the seat, and an input interface (by which information is manually inputted) is used to acquire the physical information.

In an invention according to claim 4, the health status relates to lifestyle-related diseases and to a physical condition (fatigue and fever, for example).

In an invention according to claim 5, the activity status is at least one kind among navigation information, calendar information, time information, schedule information, an activity amount of the occupant, or a difference in the daily physical information.

Further, the navigation information is at least one kind among at home, at work, at a shopping center, at a restaurant, at a fitness gym, on an expressway, on an arterial highway, in an urban area, at a hospital, or a user-setting position.

In an invention according to claim 6, the body movement sensor detects the activity amount of the occupant, and the sensor detecting the activity amount of the occupant (body movement sensor, for example) is located on at least one kind among a car key, a wristwatch, a mobile phone, a bag, a belt, and shoes.

In an invention according to claim 7, the activity status is classified into at least one kind among before work, after work, before returning home, on a business trip, before shopping, after shopping, before leisure activity, or after leisure activity.

In an invention according to claim 8, the advice relates to lifestyle and a state of the occupant.

In an invention according to claim 9, the lifestyle relates to at least one kind among diet, exercise, sleep or leisure.

In an invention according to claim 10, the advice can be used appropriately between that related to lifestyle and that related to the state of the occupant.

In an invention according to claim 11, the proper use of the advice relates to the advice for state of the occupant when before driving and the advice before driving encourages safe driving.

In an invention according to claim 12, the advice suggests activities to a subject during the day. The advice given when the occupant is returning home relates at least one kind among diet, sleep, bathing, or exercise.

In an invention according to claim 13, content of the advice changes depending on time and date, even when the health status is the same.

In an invention according to claim 14, advice reflecting previously given advice is given.

In an invention according to claim 15, the advice is notified by at least one of sound or image (character etc.).

In an invention according to claim 16, the health status is shared with at least one of doctors or family members.

In an invention according to claim 17, the shared data is transmitted wirelessly. Furthermore, the advice regarding the lifestyle of the occupant can be given to a person sharing the data (such as a spouse), based on the shared data.

In an invention according to claim 18, the advice related to diet and meal menus can be given within the automobile cabin of the occupant or the person sharing the data, based on the advice related to the occupant or the person sharing the data.

### BRIEF DESCRIPTION OF THE DRAWINGS

In the accompanying drawings:

FIG. 1 is a block diagram of an overall configuration of a health care support system according to a first embodiment;

FIG. 2 is an explanatory diagram of a health care support system disposed within a vehicle;

FIG. 3A is an explanatory diagram of an example of a configuration of physical information detecting section;

FIG. 3B is an explanatory diagram of an example of a configuration of an activity information detecting section;

FIG. 4A is an explanatory diagram of a transferring section in use;

FIG. 4B is an explanatory diagram of a notifying section;

FIG. 5 is a flowchart of a process performed by the health management support system;

FIG. 6A is an explanatory diagram of an example of physical information;

FIG. 6B is an explanatory diagram of health status judgment details;

FIG. 7 is an explanatory diagram of blood pressure abnormality judgment details;

FIG. 8A is an explanatory diagram of an example of activity status information;

FIG. 8B is an explanatory diagram of activity status judgment details;

FIG. 9 is an explanatory diagram of advice contents;

FIG. 10 is a flowchart of process details according to a second embodiment;

FIG. 11 is a flowchart of process details according to a third embodiment; and

FIG. 12 is a flowchart of process details according to a fourth embodiment.

### DETAILED DESCRIPTION OF THE PREFERRED EMBODIMENTS

Exemplary embodiments of the present invention are below described.

### First Embodiment

A health care support system according to an embodiment will be described. The health care support system measures a health status of a driver, manages information related to lifestyle-related diseases, and gives the driver advice related to the lifestyle-related diseases. The driver serves as an occupant.

a) First, an overall configuration of the health care support system according to the embodiment will be described.

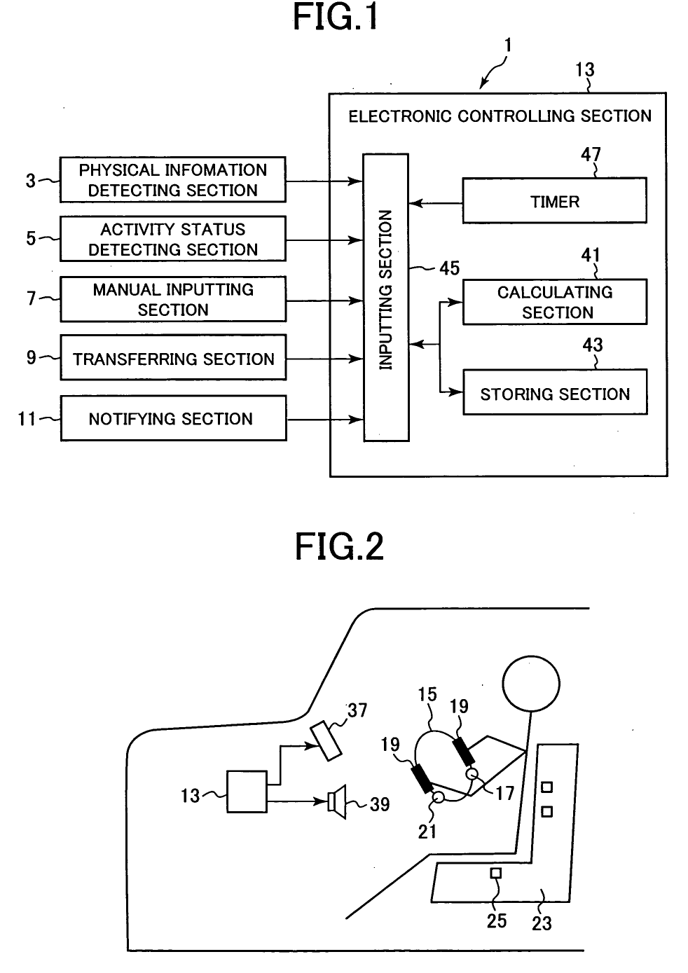

As shown in FIG. 1, a health care support system 1 includes a physical information detecting section 3, an activity status detecting section 5, a manual inputting section 7, a transferring section 9, a notifying section 11, and an electrical controlling device 13.

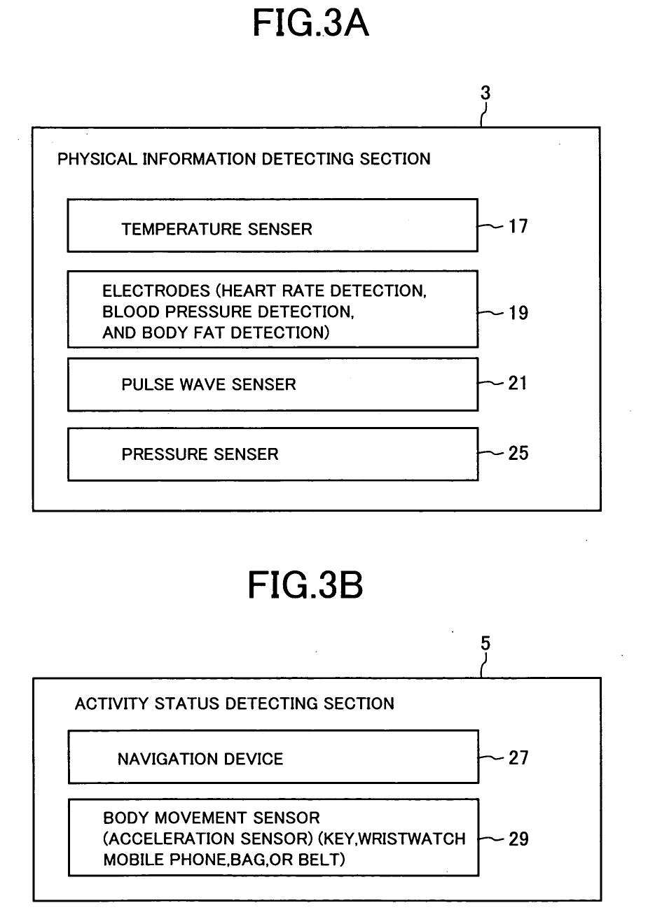

The physical information detecting section 3 is a device that measures physical information of the occupant (driver). As shown in FIG. 2 and FIG. 3A, the physical information detecting section 3 is, for example, a temperature sensor 17 disposed on a steering wheel 15, a pair of electrodes 19 attached to the steering wheel 15 or the like, a pulse wave sensor 21 attached to the steering wheel 15, and/or a pressure sensor 35 disposed on a seat 23.

Among these, the pair of electrodes 19 are used as electrodes of an electrocardiograph that acquires cardiographic signals. The electrodes 19 are also used as electrodes of a body fat scale that releases electrical current to acquire a body fat percentage. Here, the electrodes 19 and the electrical controlling device 13 function as the electrocardiograph and the body fat scale. The pair of electrodes 19 can be disposed on both left- and right-hand side edges of the steering wheel 15. Alternatively, one electrode 19 can be disposed on the steering wheel 15 and the other electrode 19 can be disposed on the seat 23.

Therefore, through use of these sensors, body temperature, blood pressure, heart rate, pulse wave information, body weight, and body fat can be detected. Specifically, the temperature sensor 17 detects the body temperature of the driver. The blood pressure is detected through a known method, from pulse wave signals (from the pulse wave sensor 21) and the cardiographic signals (refer to Japanese Patent Laid-open Publication No. 10-295656, Japanese Patent Laid-open Publication No. 10-295657, and the like). The heart rate is detected using a difference (generated by heartbeats) in electric potential (cardiographic signals) between the pair of electrodes 19. The pulse wave sensor 21 detects the pulse wave information. The pressure sensor 25 detects the body weight. The body fat can be detected from an electrical current value and the body weight. An electrical current being sent between the pair of electrodes 19 obtains the electrical current value. Blood sugar level can be detected based on, for example, optically determined glucose level (refer to Japanese Patent Laid-open Publication No. 2002-162353). Height can be manually entered.

The activity status-detecting section 5 is a device that detects information on an activity status of the driver (activity information). As shown in FIG. 3B, the activity status detecting section 5 is a navigation device 27, a body movement sensor 29 that detects body movement (activity amount), and the like. The body movement sensor 29 is, for example, an acceleration sensor.

Therefore, the navigation device 27 indicates that a vehicle (and therefore, the driver) is positioned at home, at work, at a shopping center, at a restaurant, at a fitness gym, on an expressway, on an arterial highway, in an urban area, at a hospital, or a user-setting position.

The body movement sensor 29 can be attached to a removable automobile component such as a car key, a wristwatch, a mobile phone, a bag, a belt, shoes, and the like.

The activity status is classified into before work, after work, after returning home, on a business trip, before shopping, after shopping, before leisure activity, and after leisure activity. The activity status can be determined through, for example, schedule information, navigation information, and calendar information (including time).

The manual inputting section 7 is a device through which the driver and the like manually enter data, such as the physical information and the activity status, into the electrical controlling device 13.

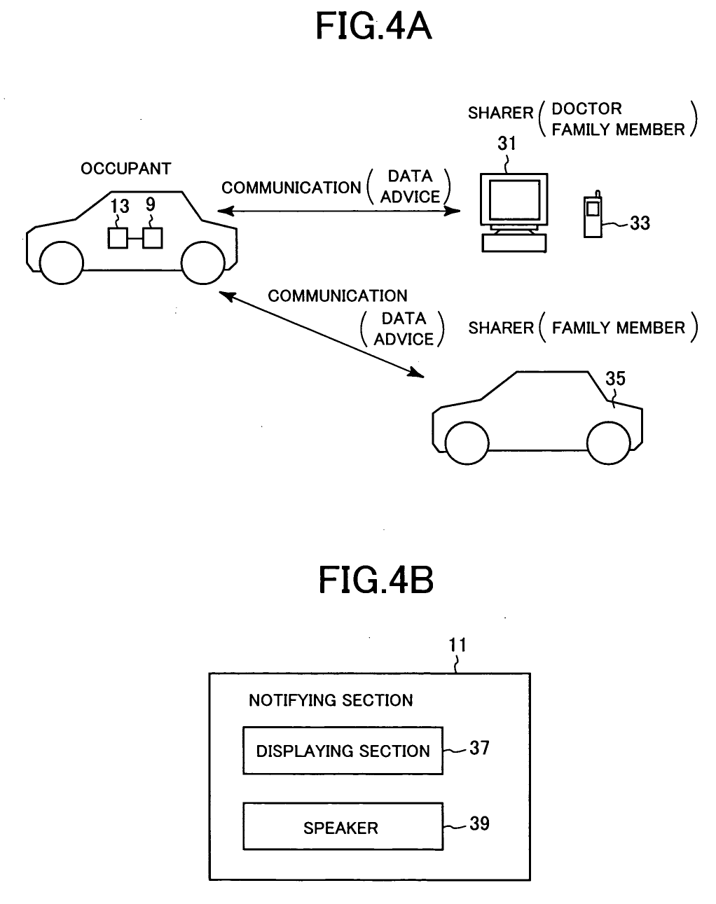

As shown in FIG. 4A, the transferring section 9 is a device that wirelessly transmits data from the electrical controlling device 13 to another device, such as a personal computer 31 at home, a mobile phone 33, or another vehicle 35. The transferring section 9 can also receive communication from an outside source.

The notifying section 11 is a device that notifies the driver and the like of advice and the like. As shown in FIG. 4B, the notifying section 11 can be, for example, a displaying section 37 such as a display, and a speaker 39.

As shown in FIG. 1, the electrical controlling device 13 is a device of which a main component is a known microcomputer. The electrical controlling device 13 includes a calculating section 41, a storing section 43, an inputting and outputting section 45, a timer 47, and the like.

The calculating section 41 performs various calculations based on inputted data and controls output of advice and the like. The storing section 43 stores various pieces of data.

The calculating section 41 functions to provide advice related to the prevention of lifestyle-related diseases, advice related to fatigue, and the like depending on the above-described physical information and activity status.

The storing section 43 stores the calendar information, the schedule information (particularly past information), and the physical information. Based on the information, differences in days of the week, information such as business trips, and normal physical information (and information acquired by the physical information detecting section) can be known. A storage device other than the electrical controlling device 13, such as a memory stick, can be used as the device storing the various pieces of information.

b) Next, details of processes performed by the health care support system will be described.

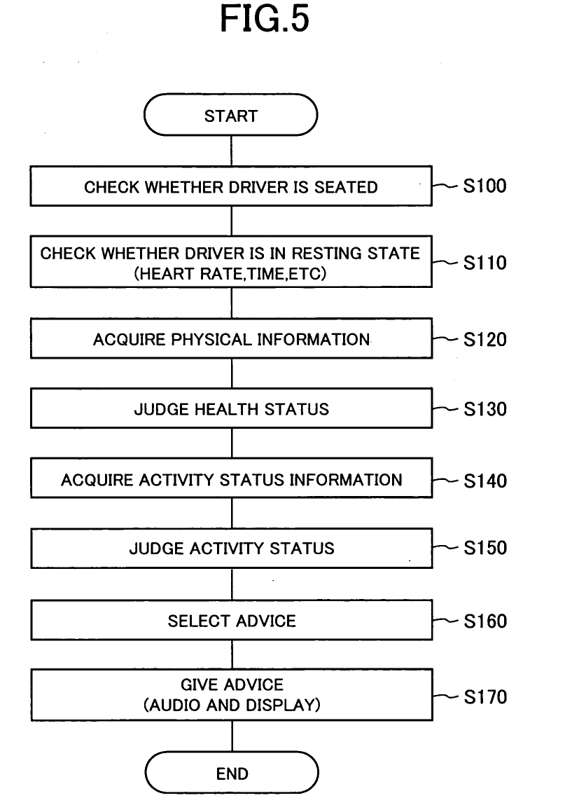

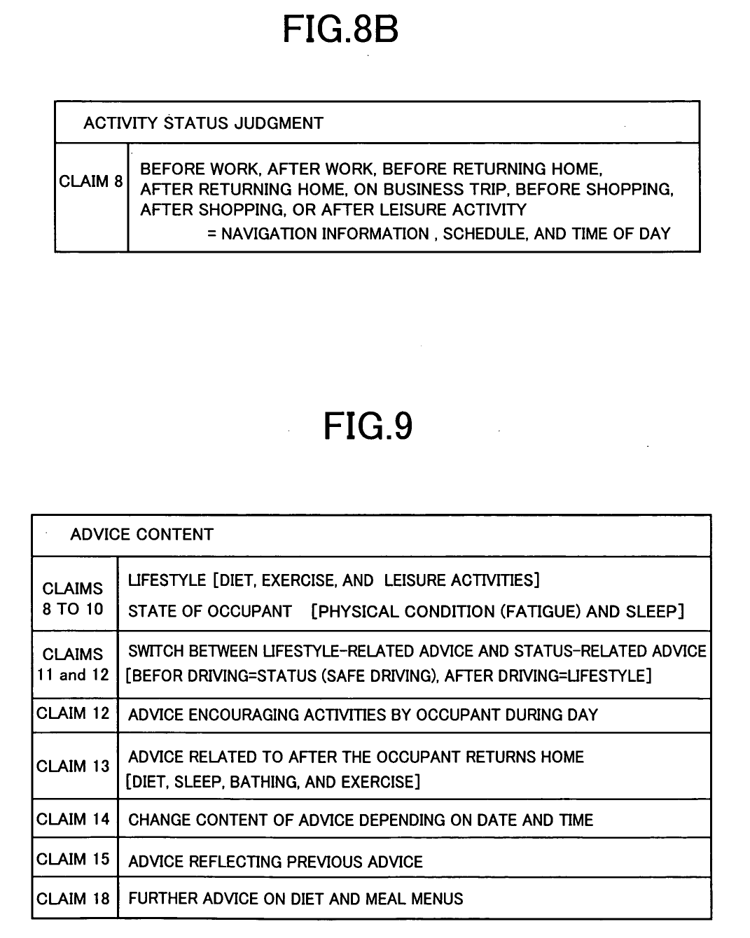

(1) First, a main routine performed by the health care support system will be described with reference to FIG. 5 to FIG. 9.

At Step (S) 100 in FIG. 5, whether the driver is seated is checked through a signal from the pressure sensor 25. When measurement does not automatically start, the measurement can be started by, for example, a manually operated measurement button.

At subsequent Step 110, because the driver is seated, whether the driver is in a resting state is checked using the heart rate acquired from the cardiographic signals by the electrodes 19, breathing information acquired from the pressure sensor 25 embedded into the seat 23, and the like. Because sharp movements are rarely made within an automobile cabin, the driver can be judged to be in the resting state after an elapse of a certain amount of time. In addition, the resting state of the driver can be judged by blood pressure (a decrease in blood pressure), body movement (a decrease in body movement), and the like.

At subsequent Step S120, because the driver is in the resting state, the physical information is acquired.

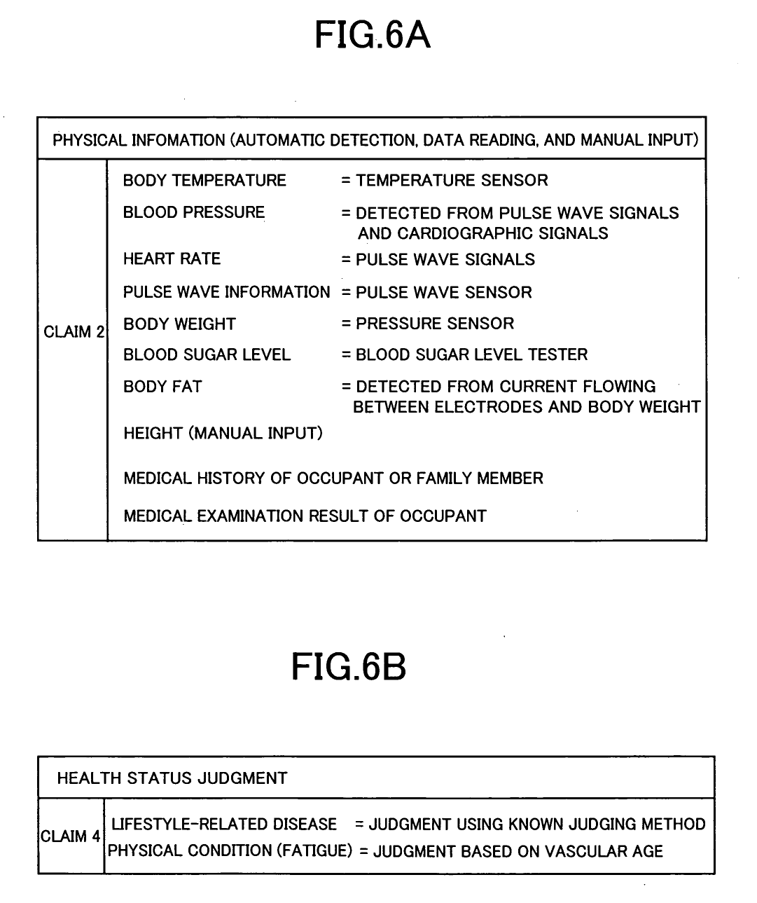

As shown in FIG. 6A, pieces of physical information are body temperature, blood pressure, heart rate, pulse wave information, body weight, blood sugar level, and body fat. The various sensors, as described above, can measure these pieces of information.

Height (manually entered) is another piece of physical information. Medical histories of passengers and family members, and medical examination results of passengers can also be entered. In addition to manual input, these pieces of information can be inputted as electronic data, wirelessly or by a portable memory (such as a memory stick) being directly connected to the health care support system 1. Through use of these pieces of physical information, accuracy of the health status judgment can be enhanced and more appropriate pieces of advice can be given.

At subsequent Step S130, the health status is judged based on the measured physical information and the like.

For example, as shown in FIG. 6B, whether a person has a lifestyle-related disease, is in a fatigued state, and the like can be judged.

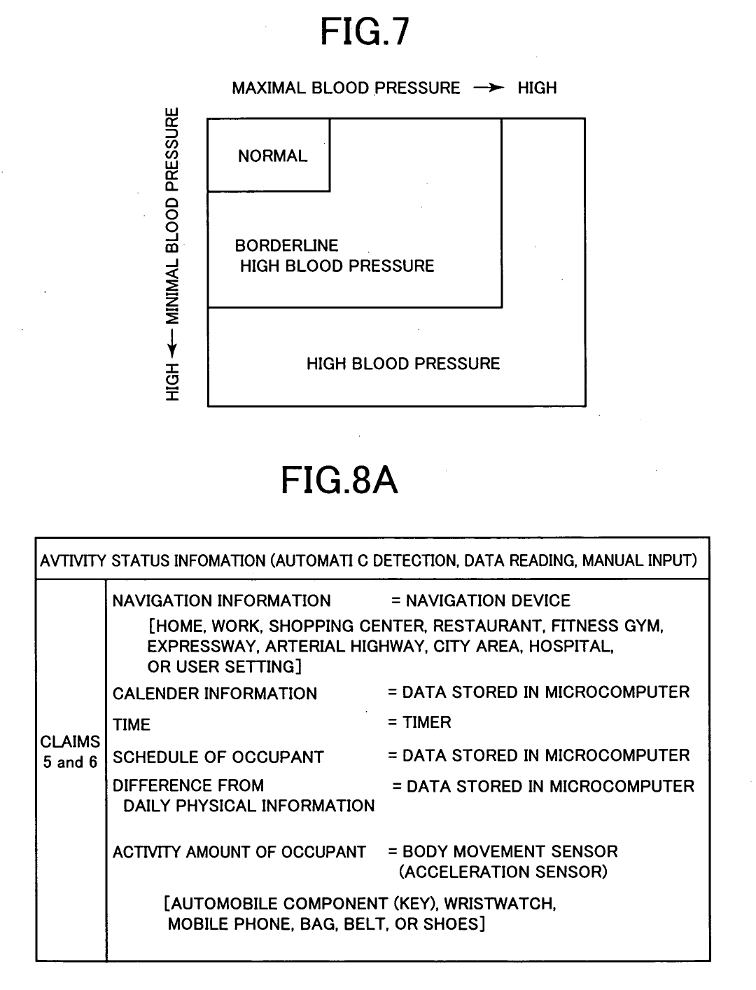

To make the judgment, a known judgment method (refer to SOM Japan website and Ministry of Health, Labor, and Welfare Kenko-Japan website) can be used. For example, as shown in FIG. 7, high blood pressure can be judged from minimal blood pressure and maximal blood pressure. Whether the body fat percentage is high or low can be judged with consideration to gender and age. Regarding heart disease, whether an abnormality is present can be judged from the measurement result from the cardiograph. Diabetes can be judged from the blood sugar level. The lifestyle-related diseases can be further judged with the addition of physical information of the driver (height and weight), medical histories of the driver and family members, and information on medical examinations can be added and.

Fatigue can be judged from changes in vascular age by determining the vascular age from, for example, a waveform that is a second-order derivative of the waveform of the driver's pulse. For example, when the vascular age is measured in the morning and in the evening and a significant difference is present between the vascular ages (the vascular age is higher in the evening), a judgment can be made that the driver is fatigued. The degree of fatigue can also be judged. Further, the existence of fever can be judged by body temperature.

At subsequent Step 140, information on activity status (activity information) is acquired.

As shown in FIG. 5A, pieces of activity information are navigation information indicating the location of the vehicle (and therefore, the driver), calendar information such as the date and the day of the week, temporal information, schedule information, and information on an activity amount.

As described above, these pieces of information can be acquired from the navigation device 27, the storing section 43, the timer 47, and the like. The information inputted by the manual can also be used. Past data (such as daily physical information) can be acquired from the storing section 43. Current activity status can be estimated from a difference in the daily physical information (such as the heart rate).

For example, the activity status of the driver can be acquired from the body movement sensor, the navigation information (for example, it is clear that the activity status is more active when the vehicle is at the gym than at work), the date information (for example, it is clear that the activity status is more active during the day than in the middle of the night; data information also allows a rough judgment of whether the activity status is before work, after work, after returning home, and the like).

At subsequent Step 150, the activity status is judged based on the activity status information.

For example, as shown in FIG. 5B, whether the activity status is before work, after work, after returning home, on a business trip, before shopping, after shopping, before leisure activity, or after leisure activity can be judged from the pieces of data, such as the navigation information, the schedule information, and the time. Therefore, by considering this activity status (place in which the occupant is present, for example), suitable advice is attained.

At subsequent Step 160, as shown in FIG. 9, for example, a piece of advice suitable for the driver or the like is selected based on the judgment result of the health status and the judgment result of the activity status.

For example, regarding lifestyle, pieces of advice related to diet (advice on food types, amounts, and the like), exercise (advice on exercise amount, degree of exercise, and the like), sleep (advice for improving irregular sleep schedules), and leisure (advice suggesting leisure activities when the amount of leisure activities is low) can be given, taking the activity status (such as the navigation information and the schedule information) into consideration.

Regarding the state of the driver, advice related to rest can be given when the driver is at home or the like and fatigued, or advice related to sleep and rest can be given when the driver is lacking sleep, taking the activity status into consideration. On the other hand, it will contribute to safe driving if advice about the stater such as fatigue, is given for example, before start driving.

Advice related to diet (advice on meal menus and the like), sleep (advice suggesting sleep and the like), bathing (advice suggesting a bath to relieve fatigue and the like), and exercise (advice suggesting exercise) can be given when the driver is returning home.

Depending on the activity status, advice can be switched between that related to lifestyle and that related to the state of the driver. For example, advice related to the state of the driver (such as advice encouraging safe driving) can be given before driving. Advice related to lifestyle (such as advice encouraging activities such as the driver taking a walk during the day) can be given after driving.

Content of the advice can be changed depending of the time and date. For example, advice suggesting a walk can be given during the day on a holiday. Advice suggesting sleep can be given at night.

Advice reflecting previous pieces of advice can also be given. For example, if advice-suggesting exercise was previously given and the driver performs strenuous exercise as a result, advice suggesting a light exercise is given next.

When the same piece of advice is given for the same health status, the occupant may grow tired of the advice. As a result, the advice may be ignored. Therefore, the content of the advice is preferably changed as required.

For example, when an advice “exercise is suggested” has been previously given, the occupant may grow tired of the same advice, as described above, when a similar advice is given again. In this case, when a certain piece of advice has been previously given, it is preferable to take the advice into consideration and give a piece of advice such as “keep up the good work” to arouse interest.

Specifically, for example, various pieces of advice such as those below can be given.

When the driver returns home (activity status) and fatigue level is high (physical information): “You are fatigued. Please rest.”

When the driver is on a holiday (activity status), activity amount for the week is low (activity status), and body fat percentage is high (physical information): “Exercise amount is low. Exercise is suggested.”

When the driver arrives at a restaurant (activity status) and body fat percentage is high (physical information): “A low-calorie meal is suggested.”

When the driver arrives at a restaurant (activity status) and blood pressure is high (physical information): “Please select a low-sodium meal.”

At subsequent Step 170, the selected piece of advice is displayed on the displaying section 37 and audibly outputted. The process is temporarily completed.

c) In this way, according to the embodiment, suitable advice can be given to the driver at an appropriate timing through the addition of the activity status in addition to physical information.

The driver receiving the advice can appropriately respond to the advice, thereby having a very positive effect on the health management of the driver himself.

### Second Embodiment

Next, a health care support system according to a second embodiment will be described. The same explanations that are used in the first embodiment are omitted.

According to the embodiment, advice is only given when the driver reaches a destination.

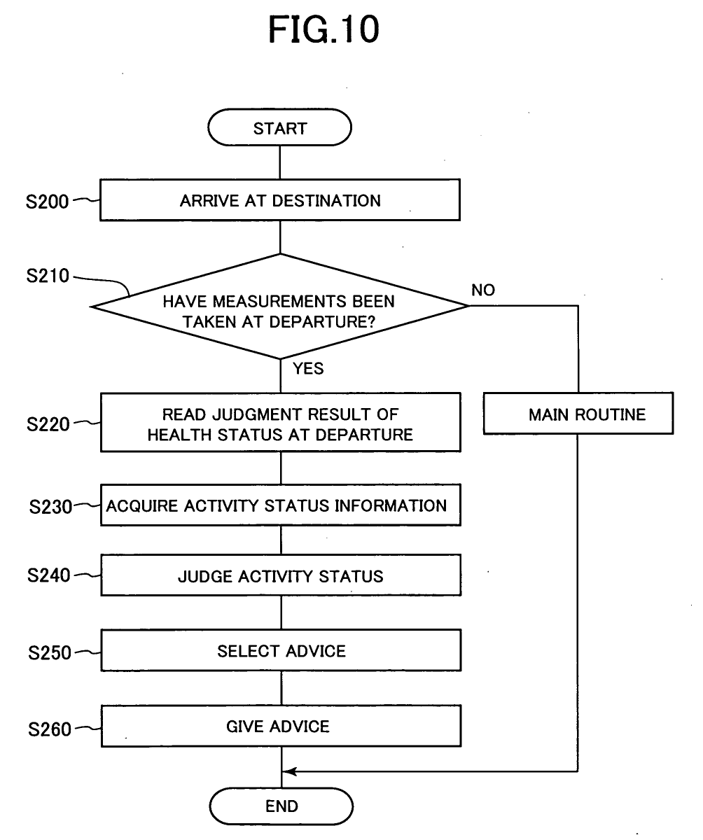

As shown in FIG. 10, at Step 200, first, arrival at the destination is checked through the navigation information and the like.

At subsequent Step 210, whether measurements (physical information) have been taken at departure is judged.

Here, when the judgment is made that the measurements have not been taken, the process at Step 110 to Step 170 is performed as in the above-described main routine and the process is temporarily completed.

At the same time, when the judgment is made that the measurements have been taken, the process proceeds to Step 220. A process for reading the physical information measured and stored in the storing section 43 at departure and the judgment results is performed (reading of only the judgment results is also possible).

At subsequent Step 230, the activity status information is acquired.

At subsequent Step 240, the activity status is judged.

At subsequent Step 250, the advice is selected based on the read judgment results of the physical information and the judgment results of the activity status after arrival.

At subsequent Step 260, the advice is given taking a most recent activity status into consideration. The process is then temporarily completed.

In this way, according to the embodiment, the advice is given based on the read judgment results of the physical information and the judgment results of the activity status after arrival. Therefore, an advantage is that processing load can be reduced.

### Third Embodiment

Next, a health care support system according to a third embodiment will be described. The same explanations that are used in the first embodiment are omitted.

According to the embodiment, when the driver gets in the car twice in a single day, second measurements (physical information) are not taken.

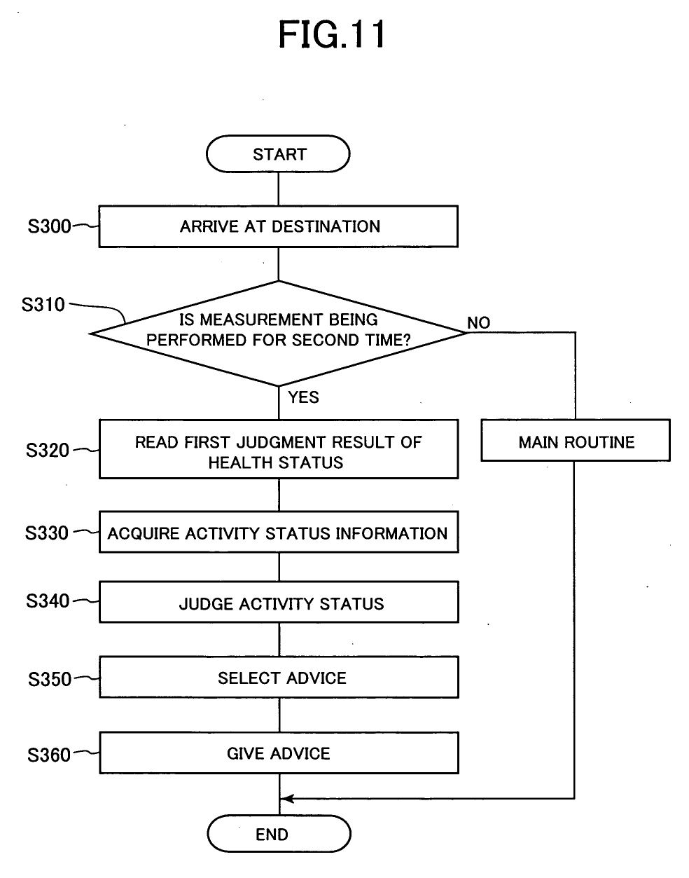

As shown in FIG. 11, at Step 300, first, whether the driver is seated is checked.

At subsequent Step 310, because the driver is seated, whether current measurements (physical information) are the second measurements to be taken on this day is judged.

When the judgment is that the current measurements are not the second measurements to be taken on this day, the process at Step 110 to Step 170 is performed as in the above-described main routine and the process is temporarily completed.

At the same time, when the judgment is that the current measurements are the second measurements to be taken on this day, the process proceeds to Step 330. A process is performed for reading the first pieces of physical information measured and stored in the storing section 43, and the judgment results (reading of only the judgment results is also possible).

At subsequent Step 330, the activity status information is acquired.

At subsequent Step 340, the activity status is judged.

At subsequent Step 350, the advice is selected based on the read judgment results of the physical information and the judgment results of the activity status after arrival.

At subsequent Step 360, the advice is given, taking a most recent activity status into consideration. The process is then temporarily completed.

In this way, according to the embodiment, the advice is given based on the judgment results of the first pieces of physical information and the judgment results of the current activity status. Therefore, an advantage is that processing load can be reduced.

### Fourth Embodiment

Next, a health care support system according to a fourth embodiment will be described. The same explanations that are used in the first embodiment are omitted.

According to the embodiment, measurements are taken twice in a single day, and fatigue and the like are judged.

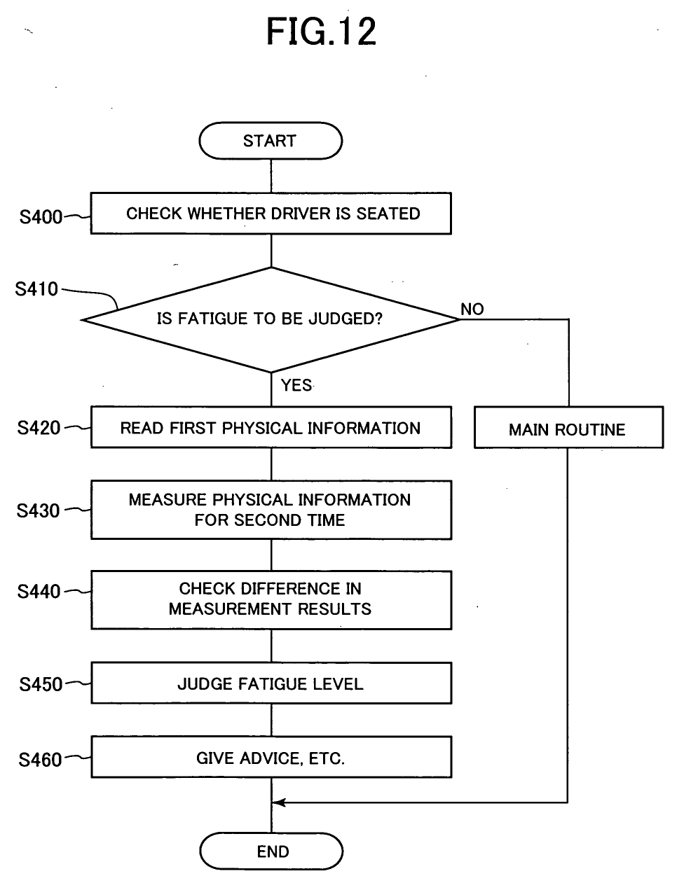

As shown in FIG. 12, at Step 400, first, whether the driver is seated is checked.

At subsequent Step 410, because the driver is seated, whether judgment regarding fatigue is to be performed at this time is judged.

When the judgment is that the judgment regarding fatigue is not to be performed at this time, the process at Step 110 to Step 170 is performed as in the above-described main routine and the process is temporarily completed.

At the same time, when the judgment is that the judgment regarding fatigue is to be performed at this time, the process for reading the first pieces of physical information (the vascular age obtained through the second-order derivative of the pulse wave) measured and stored in the storing section 43 is performed.

At subsequent Step 430, the second measurements (the vascular age in the physical information) are taken. At this time, acquisition of the activity status information and judgment of the activity status information are performed as normal.

At subsequent Step 440, the first measurement results and the second measurement results are compared.

At subsequent Step 450, the fatigue level is judged from the difference between the measurement results. For example, when the second vascular age is greater than the first vascular age by a predetermined amount or more, the fatigue level is judged to be high.

At subsequent Step 460, advice is given based on the determined fatigue level. For example, when the driver arrives home and the fatigue level is high, a piece of advice suggesting rest and the like is given. The process is temporarily completed.

In this way, according to the embodiment, advice is given based on the difference in the second physical information (such as the vascular age). Therefore, an advantage is that an appropriate piece of advice can be given depending on the fatigue level.

### Other Embodiments

(1) According to each of the above-described embodiments, various pieces of data are inputted into the electronic controlling device 13. The physical information, such as heart rate, blood pressure, and body fat percentage, are determined based on the inputted data. However, a device including a microcomputer that calculates individual pieces of physical information can be used. It is possible to input only the calculation results into the electrical controlling device 13.

(2) Various pieces of information can be wirelessly sent to doctors and family members, and data can be shared.

Advice regarding the lifestyle of the occupant can be given to a person sharing the data (such as a spouse), based on the shared data. Advice related to diet and meal menus can be given within the automobile cabin of the occupant or the person sharing the data, based on the advice related to the occupant or the person sharing the data.

For example, since a concrete menu can be proposed and the advice regarding eating habits or the menu of a meal (for example, advice which makes time of a meal regular, or advice which uses the menu of a meal as the menu for increasing vegetable intake) can be reported within vehicles. Therefore when the occupant or the like gets off the vehicles, it becomes possible to take action based on the advice. Moreover, the advice also includes a proposing of the menu; therefore checking the menu within the vehicles helps efficient shopping.

The present invention is not limited to the above-described embodiments. Various modifications can be made without departing from the scope of the invention.

What is claimed is:

1. A health care support system that is mounted on an automobile and supports a health status of an occupant, comprising:
physical information detecting means that detects a physical status of the occupant as physical information;
health status judging means that judges the health status of the occupant from the physical information;
activity status detecting means that detects activity information indicating an activity status of the occupant;
activity status judging means that judges the activity status of the occupant from the activity information;
advice selecting means that selects a piece of advice to be given to the occupant based on a judgment result acquired by the health status judging means and a judgment result acquired by the activity status judging means; and
notification controlling means that drives a notifying means and gives notification of the piece of advice.

2. The health care support system according to claim 1, wherein the physical information is at least one kind among body temperature, blood pressure, heart rate, pulse wave information, body weight, blood sugar level, body fat, and height;
the physical information includes at least one of either a medical history of the occupant or a medical history of a family member; and
the physical information includes a medical examination result of the occupant.

3. The health care support system according to claim 1, wherein at least one kind among an electrode attached to a steering wheel, a pulse wave sensor, an electrode attached to a seat, a pressure sensor attached to the seat, and an input interface is used to acquire the physical information.

4. The health care support system according to claim 1, wherein the health status relates to lifestyle-related diseases and to a physical condition.

5. The health care support system according to claim 1, wherein the activity status is at least one kind among navigation information, calendar information, time information, schedule information, an activity amount of the occupant, or a difference in the daily physical information; and
the navigation information is positioned at least one kind among at home, at work, at a shopping center, at a restaurant, at a fitness gym, on an expressway, on an arterial highway, in an urban area, at a hospital, or a user-setting position.

6. The health care support system according to claim 5, wherein a body movement sensor detects the activity amount of the occupant; and
the sensor detecting the activity amount of the occupant is located on at least one kind among a car key, a wristwatch, a mobile phone, a bag, a belt, and shoes.

7. The health care support system according to claim 1, wherein the activity status is classified into at least one kind among before work, after work, before returning home, on a business trip, before shopping, after shopping, before leisure activity, or after leisure activity.

8. The health care support system according to claim 1, wherein the advice relates to lifestyle and the state of the occupant.

9. The health care support system according to claim 8, wherein the lifestyle relates to at least one kind among diet, exercise, sleep or leisure.

10. The health care support system according to claim 1, wherein the advice can be used appropriately between that related to lifestyle and that related to the state of the occupant.

11. The health care support system according to claim 10, wherein the proper use of the advice relates to the advice for state of the occupant when before driving; and
the advice before driving encourages safe driving.

12. The health care support system according to claim 11, wherein the advice suggests activities to a subject during the day; and
the advice given when the occupant is returning home relates at least one kind among diet, sleeps, bathing, or exercise.

13. The health care support system according to claim 1, wherein content of the advice changes depending on time and date, even when the health status is the same.

14. The health care support system according to claim 1, wherein an advice reflecting a previously given advice is given.

15. The health care support system according to claim 1, wherein the advice is notified by at least one of sound or image.

16. The health care support system according to claim 1, wherein the health status is shared with at least one of doctors or family members.

17. The health care support system according to claim 16, wherein the shared data is transmitted wirelessly; and
the advice regarding the lifestyle of the occupant can be given to a person sharing the data, based on the shared data.

18. The health care support system according to claim 17, wherein the advice related to diet and meal menus can be given within the automobile cabin of the occupant or the person sharing the data, based on the advice related to the occupant or the person sharing the data.
